(19) United States
(12) Patent Application Publication
(10) Pub. No.: US 2020/0224179 A1
(43) Pub. Date: Jul. 16, 2020

(54) Carotenoid oxygenase and its application

(71) Applicant: OCEAN UNIVERSITY OF CHINA, Qingdao (CN)

(72) Inventors: Xiaoli Hu, Qingdao (CN); Zhenmin Bao, Qingdao (CN); Huizhen Wang, Qingdao (CN); Shuyue Wang, Qingdao (CN); Xue Li, Qingdao (CN); Liang Zhao, Qingdao (CN); Mengran Zhang, Qingdao (CN); Shanshan Lian, Qinghai (CN); Tingting Li, Qinghai (CN); Wei Wu, Qinghai (CN); Shi Wang, Qinghai (CN); Shiqi Liu, Qinghai (CN); Lingling Zhang, Qinghai (CN); Wei Lu, Qinghai (CN)

(73) Assignee: OCEAN UNIVERSITY OF CHINA

(21) Appl. No.: 16/828,980

(22) Filed: Mar. 25, 2020

(30) Foreign Application Priority Data
Mar. 25, 2019 (CN) 201910225151X
Mar. 25, 2019 (CN) 2019102251562

(51) Int. Cl. C12N 9/02; C12N 15/52

(52) U.S. Cl. CPC C12N 9/0069; C12Y 113/11051; C12Y 113/11065; C12N 15/52

(57) ABSTRACT

A protease with a function of degrading carotenoids of scallop is provided, which belongs to the technical field of genetic engineering. In particular, the present invention relates to the sequence and application of a carotenoid oxidase gene of scallop. The present invention analyzes the gene sequence, and verifies that the gene has function of degrading carotenoids of scallop scallop by RNA interference technology, and can be used to increase the carotenoid content of the scallop muscle of scallop scallop. The gene verified that the gene expression products can degrade carotenoids accumulated in bacteria. In addition, the RNA interference method provides a powerful tool for the study of scallop functional genes; the prokaryotic expression and protein purification methods provided can efficiently prepare oxidative lyases of carotenoid in scallop, so as to provide necessary experimental tools for in-depth research of the mechanism of shell carotene metabolism.

### CROSS REFERENCE OF RELATED APPLICATION

The present application claims priority under 35 U.S.C. 119(a-d) to CN201910225151X, filed Mar. 25, 2019, and CN2019102251562, filed Mar. 25, 2019.

### BACKGROUND OF THE PRESENT INVENTION

### Field of Invention

The invention belongs to the technical field of biological engineering, and particularly relates to a scallop carotenoid oxygenase gene, and its application.

### Description of Related Arts

Yesso scallop (Patinopecten yessoensis) was introduced into China from Japan in the 1980s and has become an important marine aquaculture shellfish in northern China. The adductor muscle of normal scallops is white, but some individuals from cultured Yesso scallop in China have orange adductor muscles. Studies have shown that carotenoids, pectenolone and pectenoxanthin, are accumulated in orangeadductor muscles, while pectenolone and pectenoxanthin are not detected in the white adductor muscle of normal Yesso scallops. Further pedigree and multiomics analysis revealed that the orange color of the adductor muscle was a recessive trait determined by a single gene, and the down-regulation of the expression of a carotenoid oxygenase gene led to the accumulation of carotenoids in the adductor muscle, which caused the orange color. This gene is a key gene for carotenoid metabolism in scallops.

Carotenoids are lipophilic yellow, orange or red natural pigments that are widely distributed in nature and have structural and functional diversity. Metabolites produced by carotenoids participate in a wide range of biological processes through oxidative degradation. For example, one of the degradation products, retinal, is a precursor of signal molecules such as 11-cis-retinal (visual chromophore) and all-trans retinoic acid.

Scallop is an important marine economic shellfish in China. Its main food is microalgae which contains a large amount of carotenoid. The preparation of scallop carotenoid oxygenase is of great significance for studying the metabolic mechanism of carotenoid in shellfish. Meanwhile, inhibiting the expression of carotenoid oxygenase could improve carotenoid content in Yesso scallop, which will be used for breeding carotenoid-enriched scallop.

### SUMMARY OF THE PRESENT INVENTION

The object of the present invention is to provide a protease for degrading scallop carotenoids and an application thereof. By inhibiting expression of the protease, the content of carotenoids in adductor muscle of the shellfish can be increased.

The present invention provides a protease for degrading carotenoids in scallop, comprising:

(1) a protease with an amino acid sequence of SEQ ID NO: 1;

(2) a homologous protease which is substituted, deleted, or added with one or more amino acids in the amino acid sequence of (1) and has protease activity of the protease in (1); and

a gene encoding the protease mentioned above, a nucleotide sequence of which is SEQ ID NO: 2;

The present invention also provides a recombinant strain of Escherichia coli (E. coli), which is utilized for recombinant expression of a protease having an amino acid sequence of SEQ ID NO: 1;

The present invention further provides a method for preparing a protease having an amino acid sequence of SEQ ID NO: 1, which is prepared by using the recombinant Escherichia coli strain mentioned above for fermentation.

The method for preparing a protease having an amino acid sequence of SEQ ID NO: 1, comprises following steps of:

step (1) constructing an expression plasmid: amplifying a coding region of the carotenoid oxygenase gene of scallop, and ligating an amplified fragment to a pET-28a expression vector;

step (2) prokaryotic expression of the protein: transforming the recombinant plasmid into competent cells of E. coli, and adding IPTG to induce protein expression; collecting the bacterial cells by centrifugation, adding a buffer solution to the collected bacterial cells, breaking cells using a sonicator; collecting the supernatant and pellet separately after centrifugation, performing SDS-PAGE detection; and

step (3) purifying of the protein: according to the results of SDS-PAGE, inducing a large amount of expression, and collecting the bacterial cells by centrifugation; lysing the bacterial cells with a buffer solution, sonicate, and then collecting the crude protein by centrifugation; purifying proteins with Ni-NTA column; respectively processing, sampling, and testing by SDS-PAGE crude protein, washed-out and eluted effluent; dialyzing better-purified components, concentrating, and filtering, and then storing at −80° C. after dispensing.

Another aspect of the present invention also provides a method for increasing the content of carotenoids, which is performed by reducing the amount of a protease whose amino acid sequence is SEQ ID NO: 1;

The reduced amino acid sequence is a protease of SEQ ID NO: 1, and one way thereof is accomplished by means of RNAi;

One specific step of the method is as follows:

(1) synthesis of double-stranded RNA (dsRNA):

synthesizing a double-stranded RNA capable of reducing the gene expression of the nucleotide sequence of SEQ ID NO: 2;

(2) injecting dsRNA into shellfish to interfere the expression of the target gene, thereby reducing the expression of protease.

The present invention uses genetic engineering technology to clone the coding region of the carotenoid oxygenase gene into a prokaryotic expression vector, and then transforms the recombinant plasmid into E. coli, and uses isopropyl-β-D-thiogalactopyranoside (IPTG) to induce the expression of fusion protein. The fusion protein was purified, and concentrated by dialysis. The prokaryotic expression and protein purification method provided by the present invention can efficiently prepare scallop carotenoid oxygenase, and provide necessary experimental tools for in-depth study of mechanism of carotenoids metabolism in shellfish carotene metabolism at the protein level.

These and other objectives, features, and advantages of the present invention will become apparent from the following detailed description, the accompanying drawings, and the appended claims.

### BRIEF DESCRIPTION OF THE DRAWINGS

FIG. 1: HPLC detection of carotenoids in the adductor muscle of the experimental and control groups. A: experimental group with dsRNA injection for inhibiting carotenoid oxygenase expression, B: control group.

FIG. 2: SDS-PAGE analysis of prokaryotic expression of the fusion protein. M: Protein Marker; 1: total protein from the cells without induction of carotenoid oxygenase expression; 2: supernatant of lysis of E. coli cultured at 20° C. for inducing carotenoid oxygenase expression; 3: pellets of lysis of E. coli cultured at 20° C. for inducing carotenoid oxygenase expression; 4: supernatant of lysis of E. coli cultured at 37° C. for inducing carotenoid oxygenase expression; 5: pellets of lysis of E. coli cultured at 37° C. for inducing carotenoid oxygenase expression.

FIG. 3: Color change of β-carotene accumulating strains caused by expression of carotenoid oxygenase, (C−): β-carotene accumulating strains with empty plasmid pET-28a; (C+): β-carotene accumulating strains with recombinant plasmid for expressing carotenoid oxygenase.

FIG. 4: SDS-PAGE analysis of fusion protein purified with nickel-agarose affinity chromatography. M: Protein marker; 1: loading; 2: elution; 3-4: 20 mM Imidazole-eluted fraction; 5: 50 mM Imidazole eluted fraction; 6: 500 mM Imidazole-eluted fraction.

FIG. 5: SDS-PAGE analysis of the final purified protein. M: Protein marker; 1: target fusion protein.

FIG. 6: Expression of carotenoid oxygenase genes in striated muscle with dsRNA injection;

FIG. 7: Color change of scallop adductor muscle with dsRNA injection. A: control group, injected with 1×PBS solution; B: experimental group, injected with dsRNA.

### DETAILED DESCRIPTION OF THE PREFERRED EMBODIMENT

The protease of the present invention is described in detail below with reference to the examples.

### Example 1: Screening for Proteases

The applicant has discovered through multiple omics studies such as whole- genome linkage analysis, association analysis, and expression profiling that the reason for the accumulation of carotenoids in scallop with orange adductor muscles is that the expression of carotenoid oxygenase is down-regulated, which reduces the degradation of carotenoids.

The sequence of carotenoid oxygenase gene was obtained from the scallop genome and transcriptome database. The gene is 9107 bp in length and the corresponding cDNA is 2682 bp in length. The CDS length is 1566 bp (SEQ ID NO: 2) and encodes 521 amino acids (SEQ ID NO: 1). The predicted molecular weight of the protein is 59.2 KDa, and the isoelectric point is 6.54. The 5′ UTR and 3′ UTR length is 510 bp and 609 bp, respectively.

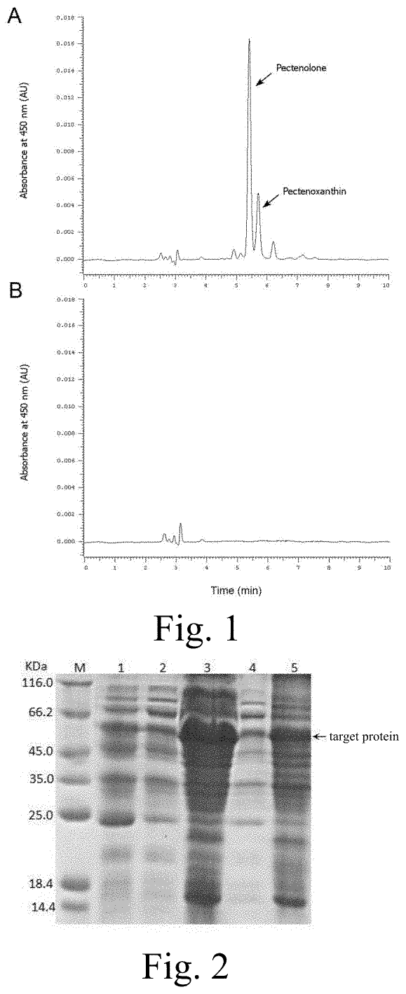

dsRNA injection was performed to inhibit the expression of carotenoid oxygenase. qRT-PCR was used to detect the effect of interference. By observing the changes in the color of the adductor muscle and the following HPLC analysis, carotenoids accumulation in the muscles of scallops with carotenoid oxygenase gene interference was determined (FIG. 1), that is, the gene identified has the function of degrading carotenoids.

Though suppressing the expression of carotenoid oxygenase gene by RNA interference (RNAi), and detecting the change in the expression of carotenoid oxygenase gene and carotenoids content of in the adductor muscle of scallop after carotenoid oxygenase gene interference, function of the analyzed gene in carotenoid cleavage is identified, and the gene encodes a carotenoid oxygenase of scallop.

### Example 2

The prokaryotic expression of the scallop carotenoid oxygenase protein in the invention includes the following steps:

a) amplification of carotenoid oxygenase coding sequence;

b) construction of expression plasmids;

c) prokaryotic expression of carotenoid oxygenase;

d) Verification of carotenoid oxygenase activity in prokaryotic systems.

The specific operations are as follows:

a) Amplification of carotenoid oxygenase coding sequence. Using the scallop striated muscle cDNA as the template, using primers containing the corresponding restriction sites and protective bases of the pET-28a vector:

5′-CCGGAATTCGTCCCACCAGC-3′;
5-TCCCAAGCTTTCAAGGGATGTTAAAGAACC-3 ′

Amplify the target fragment. The reaction system (20 μL) is:

Template cDNA 2.5 μL

5×Phusion HF Buffer 4.0 μL

10 mM dNTPs 0.4 μL

Forward primer (10 mM) 1.0 μL

Reverse primer (10 mM) 1.0 μL

Phusion DNA Polymerase 0.2 μL

ddH2O add to 20 μL

The PCR reaction program used for the amplification was: denaturation at 98° C. for 30 s; denaturation at 98° C. for 10 s, annealing at 62° C. for 30 s, extension at 72° C. for 50 s, 30 cycles; extension at 72° C. for 10 min.

b) Construction of expression plasmid. Digestion of the amplified target fragment and pET-28a vector, the digestion system is:

CutSmart® Buffer (10×) 5μL

DNA 1 μg

Ecor I 1 μL

Hind III 1 μL

ddH2O add to 50 μL

Digestion was performed at 37° C. for 15 min. The digestion products were purified by QIAquick PCR Purification Kit.

The purified fragment was ligated with the vector using T4 ligase. The ligation system (25 μL) was:

T4 DNA Ligase Buffer 2.5 μL

ATP 2 μL

Purified digestion fragment 100 ng

Vector 300 ng

T4 DNA Ligase 1 μL

Ligation was carried out with PCR instrument at 16° C. overnight. After obtaining the recombinant plasmid through transformation and extraction, sequencing was performed to verify the correctness of the introduced sequence.

c) Prokaryotic expression of carotenoid oxygenase. The recombinant plasmid was transferred into Rosetta (DE3) competent cells and spread on LB solid medium (50 μg/mL kanamycin). Single clones were picked and cultured in a liquid medium containing antibiotics. When the OD value reached 0.6, the inducer IPTG was added and the cells werecultured at 20° C. for 16 h or 37° C. for 4 h. The negative control was cultured without the inducer. Then the bacterial cells were collected by centrifugation, and the supernatant was discarded. Buffer A was added to the collected bacterial cells, and the cells were fully broken using a sonicator. Centrifugation was performed again, and the precipitate after centrifugation was dissolved in buffer B. The supernatant and the precipitate were collected separately, and subjected to SDS-PAGE detection. The optimal expression conditions were determined based on the detection results (FIG. 2).

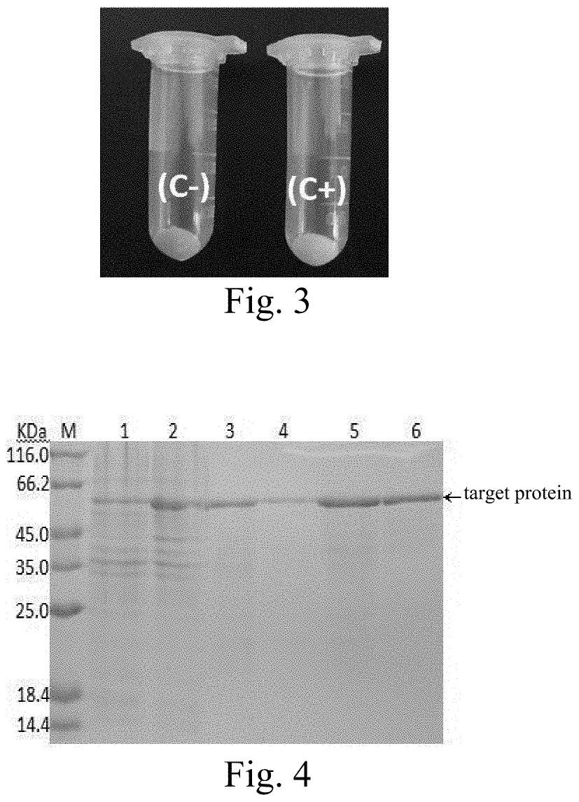

d) Verification of carotenoid oxygenase activity in prokaryotic systems. The recombinant plasmid was transformed into E. coli which could synthesize β-carotene and is in orange color. Then the E. coli cells were spread on LB solid medium (50 μg/mL kanamycin). Pick a single clone and culture it in liquid medium (50 μg/mL kanamycin, 25 μg/mL chloramphenicol) until the OD600 reaches 0.6. After adding the inducer IPTG, the culture was continued at 28° C. for 16 hours in dark. At the same time, the empty pET-28a plasmid was also transformed into E. coli which could synthesize β-carotene, and the cells were then cultured and induced in the same way as above. Cells were collected by centrifugation, and the color of the bacteria was observed. It was found that the color of the strain expressing the carotenoid oxygenase protein was obviously lighter than the strain with the empty pET-28a plasmid, which proved that the prokaryotically expressed enzyme has carotenoid oxygenase function (FIG. 3).

### Example 3

Purification of scallop carotenoid oxygenase protein in the present invention includes the following steps:

a) large amount expression of carotenoid oxygenase protein;

b) protein purification;

c) purification test;

d) dialysis concentration.

The specific operations are as follows:

a) Large amount expression of carotenoid oxygenase protein. Bacteria were cultured in a liquid medium containing 50 μg/mL kanamycin. When the OD value reached 0.6, 0.5 mM inducer IPTG was added, and the cells were cultured at 20° C. for 16 h to express a large number of cells.

b) Protein purification. The bacterial cells were lysed with buffer C and then sonicated, and the supernatant crude protein was collected by centrifugation. Take 5 mL of Ni-NTA and wash the equilibration column with 5 times the bed volume of Binding buffer. The flow rate is 5 mL/min. The crude protein was incubated with the equilibrated column packing for 1 h, and then the incubated product was loaded onto the column and collected. Then wash the equilibration column with Binding buffer, wash the column with Washing buffer, and collect the effluent. Finally the column was washed with Elution buffer and the effluent was collected.

c) Purification test. Crude protein, washed-out and eluted effluent were collected and prepared for SDS-PAGE detection (FIG. 4).

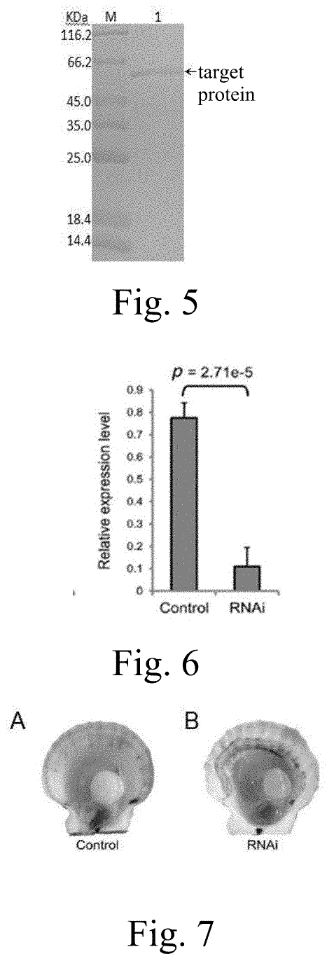

d)Dialysis concentration. Fraction 6 which showed higher purity was dialyzed into a protein storage buffer (1×PBS, 20% Glycerol, 2 mM DTT, 0.1% SKL, pH=8.0). At the end of dialysis, the protein was concentrated with PEG20000, filtered through a 0.22 μm filter membrane, and then packed and stored at −80° C. Finally, 5 mg of carotenoid oxygenase recombinant protein with a purity of >85% was obtained (FIG. 5).

The above buffer solution is formulated as follows:

| Name of buffer | Formulas |
|---|---|
| Buffer A | 50 mM Tris, 300 mM NaCl, pH = 8.0 |
| Buffer B | 8M Urea, 50 mM Tris-HCl, |
| | 300 mM NaCl, pH = 8.0 |
| Buffer C | 7M Gua-HCl, 50 mM Tris, 300 mM NaCl, |
| | 0.1% Triton X-100, pH = 8.0 |
| Binding buffer | 8M Urea, 50 mM Tris, 300 mM NaCl, pH = 8.0 |
| Washing buffer | 8M Urea, 50 mM Tris, 300 mM NaCl, |
| | 20/50 mM Imidazole, pH = 8.0 |
| Elution buffer | 8M Urea, 50 mM Tris, 300 mM NaCl, |
| | 500 mM Imidazole, pH = 8.0 |

### Example 4: Improving Carotenoids Content in Scallop by RNAi Interference

The specific operation of RNAi is as follows:

a) Primer design. Use siDirect version 2.0 (http://sidirect2.rnai.jp/) to predict and screen RNAi target sequences of genes. According to the basic principles of RNAi primer design, corresponding primers are designed for the target sequence. The T7 promoter is introduced at the 5 end of each primer. The primer sequence is:

5′-TAATACGACTCACTATAGGGCTCCCTTCGATGTAGCTGAAAAATT-
3′;
5′-TAATACGACTCACTATAGGGAGTTTGGTGACAGAGAACGAATG-3′.

b) Preparation of RNAi template. Using the scallop muscle cDNA of Yesso scallop as the template, the Phusion high-fidelity DNA polymerase was used to amplify the PCR product with T7 promoter. After the product was purified by QIAquick PCR Purification Kit, its quality was detected by 1.5% agarose gel electrophoresis, and was stored at −20° C. as a template for subsequent synthesis of dsRNA.

c) In vitro transcription and synthesis of dsRNA. The MEGAscript® RNAi Kit is used for in vitro transcription reaction to prepare dsRNA. The reagents and equipment used are processed to avoid nuclease. The reaction system (20 μL) is as follows: template DNA 3 μg, T7 Reaction Buffer (10×) 2 μL, ATP solution (75 mM) 2 μL, CTP solution (75 mM) 2 μL, GTP solution (75 mM) 2 μL, UTP solution (75 mM) 2 μL, T7 Enzyme Mix 2 μL, Nuclease-free Water for supplement. Add all reagents on ice, and mix slightly after centrifugation. The mixture was then incubated at 37° C. for 6 h, and then at 75° C. for 5 min, followed by cooling to room temperature to maximize the amount of product. The DNA and ssRNA were digested with nuclease. The reaction system was: 20 μL of the product from the previous step, 5 μL of Digestion Buffer (10×), 2 μL of DNase I, 2 μL of RNase, and supplemented with nuclease-free Water to 50 uL. The above reagents were added in the tube on ice, mixed and centrifuged, and incubated at 37° C. for 1 h. DsRNA was purified using water-saturated phenol and chloroform: isoamyl alcohol (24: 1, v/v). Precipitate dsRNA with isopropanol (−20° C. pre-cooled) and sodium acetate solution (4° C. pre-cooled), then wash the precipitate twice with 75% ethanol (pre-cooled at −20° C.). 50 μL of 1×PBS solution (Nuclease-free) was used to dissolve the pellet. The quality of dsRNA was measured by 1.5% agarose gel electrophoresis, and its concentration was measured by Nanoview (GE).

d) Injection of dsRNA for interfereing the expression of carotenoid oxygenase gene. Scallops were divided into two groups, namely the experimental group and the control group. Dilute dsRNA to 1 μg/μL with 1×PBS. Use a micro syringe to inject 100 μL of the diluted dsRNA solution into the adductor muscle of the scallop in the experimental group. Inject 100 μL 1×PBS into the control scallop in the same way. In the adductor muscle. The above experimental steps were performed once a week for a total of four injections. Throughout the experiment, two groups of scallops were reared under exactly the same conditions, and Chlorella was fed twice a day. After the experiment, the color of the adductor muscle was observed. The adductor muscle of the experimental group turned orange (FIG. 6).

e) The effect of qRT-PCR on interference detection. Two groups of adductor muscle of scallops were obtained by dissection, and frozen in liquid nitrogen and stored at −80° C. RNA was extracted and reverse-transcribed to obtain cDNA which was diluted 20-fold as the template. Design primers for qRT-PCR experiments of the carotenoid oxygenase gene, and select Ubiquitin (UBQ) as the internal reference gene. The primer sequence is as follows:

Target gene upstream primer:
5′-GATGCCAGGCTCTAAAGCAAC-3′;
Target gene downstream primer:
5′-CTGAACCCGTACGAGTAACGATACT-3′;
Ubiquitin-s
5′-TCGCTGTAGTCTCCAGGATTGC-3′;
Ubiquitin-a
5′-TCGCCACATACCCTCCCAC-3′.

The qRT-PCR reaction system is as follows: cDNA template 2 μL, primer-s (2 μM) 4 μL, primer-a (2 μM) 4 μL, SYBR Green I Real-time PCR Master Mix (2×) 10 μL. Each sample was set up in triplicate, and the reaction was performed on the LightCycler 480 Real-time PCR System. The program was set to: 50° C., 2 min; 94° C., 10 min; 94° C., 15 s, and 62° C. 1 min, 40 cycles. After the amplification reaction is complete, a fusion curve analysis is performed to exclude the effects of non-specific amplification. The test results showed that the expression level of the target gene in the scallop muscle of the experimental group was significantly reduced (FIG. 7).

f) Determination of carotenoid content by HPLC. Accurately weigh the adductor muscle of scallop, and the same weight of muscle was lyophilized for each sample. To prevent carotenoids from being oxidized, 0.1% BHT was added to methanol as an extraction solution. The extract was added to the lyophilized tissue, and the mixture was homogenized, centrifuged for 1 min (13,000 rpm, 4° C.) and the supernatant was collected. The above extraction steps were repeated three times, and the supernatants were combined and then subjected to rotary evaporation. Dry the residual liquid with nitrogen, then use 2 mL of mobile phase to dissolve the carotenoids, pass through a 0.22 pm filter, and load them into an autosampler vial. The above steps are performed in dark conditions, taking care to keep low temperature. LaChrom C18 (4.6×250 mm, 5 μm ID) column (HITACHI, Japan) was used to analyze the samples. The mobile phase was acetonitrile: methanol: dichloromethane (50: 46: 4, v/v/v) and 0.1. % BHT. The HPLC parameters are as follows: injection volume is 10 μL, column temperature is 25° C., flow rate is 1.0 mL/min, monitoring wavelength is 450 nm, and separation time is 10 min.

The chromatographic peaks of pectenolone and pectenoxanthin were identified based on the retention time. The results showed that carotenoids were detected in the RNAi group samples, while no carotenoid accumulation was found in the control group.

The above results indicate that by reducing the amount of the protease having the amino acid sequence of SEQ ID NO: 1, the carotenoid content in scallop can be improved.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A protease, comprising:
(1) a protease with an amino acid sequence of SEQ ID NO: 1;
(2) a homologous protease which is substituted, deleted, or added with one or more amino acids in the amino acid sequence of (1) and has protease activity of the protease in (1); and
(3) an allele enzyme of the protease with the amino acid sequence of the SEQ ID NO: 1, which is screened from Patinopecten yessoensis.

2. A method for cleaving carotenoids comprising introducing the protease according to claim 1.

3. A method for increasing carotenoids content in animals, through reducing a content of the protein as recited in claim 1.

4. The method, as recited in claim 3, which is implemented by reducing an expression amount of a gene encoding the protease according to claim 1.

5. The method, as recited in claim 4, wherein a nucleotide sequence of the encoded gene is SEQ ID NO: 2.

6. The method, as recited in claim 3, wherein the animal is an aquatic organism.

7. The method, as recited in claim 6, wherein the aquatic organism is a mollusk, an arthropod, an echinoderm or algae.

8. The method, as recited in claim 7, wherein the mollusk is shellfish, the arthropod is shrimp and crab, and the echinoderm is sea urchin.

9. The method, as recited in claim 8, wherein the shellfish is bivalve shellfish.

10. The method, as recited in claim 9, wherein the bivalve shellfish is scallop.
